(19) United States
(12) Patent Application Publication
(10) Pub. No.: US 2012/0213765 A1
(43) Pub. Date: Aug. 23, 2012

(54) METHOD AND COMPOSITION MITIGATING FECAL AND FLATULENT ODORS

(75) Inventors: RANDOLPH FLETCHER SPAINHOUR, BURGAW, NC (US); VAUGHN MATTHEW KING, RALEIGH, NC (US)

(73) Assignee: COWKIDS PET PRODUCTS, LLC, RALEIGH, NC (US)

(21) Appl. No.: 13/335,909

(22) Filed: Dec. 22, 2011

Related U.S. Application Data
(60) Provisional application No. 61/425,849, filed on Dec. 22, 2010.

(51) Int. Cl. A61K 33/44; A61P 1/00

(52) U.S. Cl. 424/125

(57) ABSTRACT

A method of mitigating or eliminating the odor associated with the feces or flatulence of an animate subject, whether human or animal, by means of administering activated carbon, whether alone or in composition with further inert ingredients, to the subject for ingestion and digestion of the same in advance of the subject either defecating or expelling flatulence.

### CROSS-REFERENCE TO RELATED APPLICATIONS

This application claims the benefit of U.S. Provisional Application Ser. No. 61/425,849 filed Dec. 22, 2010, which is hereby incorporated by reference in its entirety.

### BRIEF DESCRIPTION OF THE SEVERAL VIEWS OF THE DRAWING

Applicant asserts that a drawing of his inventive method, as disclosed above and claimed below, is unnecessary for the understanding of the method sought to be patented and, therefore, is not required per 37 C.F.R. §1.81.

Further, applicant asserts that a drawing of his inventive composition, as disclosed above and claimed below, is unnecessary for the understanding of the method sought to be patented and, therefore, is not required per 37 C.F.R. §1.81.

### WRITTEN DESCRIPTION OF THE INVENTION

The first disclosed invention is a method of mitigating or eliminating the odor associated with the feces or flatulence of an animate subject, whether human or animal, by means of administering activated carbon, whether alone or in composition with further inert ingredients, to the subject for ingestion and digestion of the same in advance of the subject either defecating or expelling flatulence.

It is contemplated that the manner and process of using the above disclosed method invention would entail a human or animal consuming either (i) pure activated carbon or (ii) activated carbon in composition with inert ingredients that may enhance the flavor, consistency, or overall appeal of the activated carbon for ingestion. Upon digestion of the same by the human or animal subject, the activated carbon, due in part to known adsorption properties, has the effect of mitigating or eliminating the odors associated with the feces and flatulence of the human or animal subject.

It is a preferred embodiment of the contemplated inventive method wherein activated carbon in composition with inert ingredients comprising an edible binding agent and selected flavoring components are orally administered to an animal in an edible biscuit form. In this case a user would feed the contemplated composition in biscuit form to an animal otherwise anticipated to produce foul smelling odors via bowel movement or just flatulence. The expected result is that the animal subject will then engage in a bowel movement or acts of flatulence, said actions failing to produce foul smelling odors normally associated with the same.

The best mode of this first disclosed invention contemplated by the inventors is the method, as described, wherein an animal ingests activated charcoal, said activated charcoal being administered to said animal by means of an edible treat comprising activated charcoal in combination with inert ingredients gelatin powder, as the binding agent, and any of a host of flavoring components commonly known to be appealing to humans and animals alike.

While the foregoing written description of the inventive method enables one of ordinary skill to make and use what is considered presently to be the best modes thereof, those of ordinary skill will understand and appreciate the existence of variations, combinations, and equivalents of the specified embodiments, methods, and examples herein. The invention should therefore not be limited by the above described embodiment, method, and examples, but shall include all embodiments and methods within the scope and spirit of the invention as claimed.

A second disclosed invention is a composition of matter contemplated by the above disclosed method invention, said composition of matter comprising activated carbon in combination with at least one inert ingredient selected for its properties either aiding in the ingestion of the activated carbon or otherwise increasing the appeal of ingesting the activated carbon to the animate subject.

It is contemplated that the manner and process of making this disclosed inventive composition of matter would be such that the activated carbon is in combination with at least one inert ingredient selected based upon flavor or consistency properties intended to make the overall composition of matter more appealing as an edible composition, whether to be eaten by human or animal. Further, it is contemplated that the composition will be produced in a multitude of individual units that may each be individually ingested.

It is contemplated that the manner and process of using this disclosed inventive composition would entail ingestion of an individual unit of the composition by an animate subject, whether human or animal, and digestion of the same for the purpose of allowing the adsorption properties of activated carbon to act within the digestive tract of the subject, resulting in the mitigation or elimination of the odor associated with the feces or flatulence of the subject.

It is a preferred embodiment of the contemplated inventive composition wherein the activated carbon is in the form of activated charcoal in composition with inert ingredients comprising an edible binding agent and selected flavoring components in an edible biscuit form. In this case a user would feed the contemplated composition in biscuit form to an animal otherwise anticipated to produce foul smelling odors via bowel movement or just flatulence. The expected result is that the animal subject will then engage in a bowel movement or acts of flatulence, said actions failing to produce foul smelling odors normally associated with the same.

The best mode of this second disclosed invention contemplated by the inventors is the composition of matter, as described, wherein the activated carbon is in the form of activated charcoal, and said activated charcoal is in combination with inert ingredients gelatin powder, as the binding agent, and any of a host of flavoring components commonly known to be appealing to humans and animals alike.

While the foregoing written description of the invention enables one of ordinary skill to make and use what is considered presently to be the best modes thereof, those of ordinary skill will understand and appreciate the existence of variations, combinations, and equivalents of the specified embodiments, methods, and examples herein. The invention should therefore not be limited by the above described embodiment, method, and examples, but shall include all embodiments and methods within the scope and spirit of the invention as claimed.

What is claimed is:

1. A method comprising introducing into the digestive tract of an organism of the kingdom Animalia a selected concentration of activated carbon for the purpose of mitigating foul smelling odors generated within the digestive tract.

2. The method of claim 1 wherein the organism of the kingdom Animalia is also of the class Mamalia.

3. The method of claim 1 wherein the activated carbon is in the form of activated charcoal.

4. The method of claim 2 wherein the organism of the class Mamalia is a human.

5. The method of claim 2 wherein the organism of the class Mamalia is not a human.

6. The method of claim 5 wherein the organism of the class Mamalia is a cat.

7. The method of claim 5 wherein the organism of the class Mamalia is a dog.

8. The method of claim 1 wherein the introduction into the digestive tract of the organism is by way of ingestion.

9. A composition that mitigates foul smelling odors generated within the digestive tracts of organisms of the kingdom Animalia, the composition comprising a selected concentration of activated carbon in combination with at least one inert component selected for its appeal among said organisms in making the overall composition more appealing to ingest.

10. The composition of claim 9 wherein the activated carbon is in the form of activated charcoal.

11. The composition of claim 9 wherein at least one inert component is an edible binding agent.

12. The composition of claim 11 wherein at least one edible binding agent is gelatin.

13. The composition of claim 11 wherein at least one secondary inert component is an edible flavoring agent.

14. The composition of claim 9 wherein at least one inert component is an edible flavoring agent.
